(19) United States
(12) Patent Application Publication
(10) Pub. No.: US 2025/0186032 A1
(43) Pub. Date: Jun. 12, 2025

(54) SURGICAL SMOKE FILTERING DEVICE

(71) Applicant: Covidien LP, Mansfield, MA (US)

(72) Inventor: Henry E. Holsten, Sumter, SC (US)

(21) Appl. No.: 18/844,457

(22) PCT Filed: Mar. 8, 2023
(86) PCT No.: PCT/IB2023/052198

§ 371 Date: Sep. 6, 2024

Related U.S. Application Data
(60) Provisional application No. 63/318,315, filed on Mar. 9, 2022.

(51) Int. Cl. A61B 17/02; A61B 17/00

(52) U.S. Cl. CPC A61B 17/0218; A61B 2017/00367; A61B 2017/00561

(57) ABSTRACT

A surgical smoke filtering device includes an annular collar defining a longitudinally-extending central passageway configured to be placed over an incision. A plurality of apertures is disposed through the annular collar and is configured to receive surgical smoke therethrough. A first electrode and a second electrode are disposed within the annular collar. The first electrode has a first electrical charge, and the second electrode has a second electrical charge. The first and second electrodes are configured to generate an airflow to draw the surgical smoke through the annular collar.

### INTRODUCTION

The present disclosure relates to surgical procedures, and more particularly, to a smoke filtering device for placement over an open incision to filter particulates from surgical smoke generated at an internal surgical site.

### BACKGROUND

In minimally-invasive surgical procedures, operations are carried out within an internal body cavity through small entrance openings in the body. The entrance openings may be natural passageways of the body or may be surgically created, for example, by making a small incision into which an access device is inserted.

Minimally-invasive surgical procedures may be used for partial or total removal of tissue from an internal body cavity. However, the restricted access provided by minimally-invasive openings (natural passageways and/or surgically created openings) presents challenges with respect to maneuverability and visualization. The restricted access also presents challenges when large tissue specimens are required to be removed. As such, tissue specimens that are deemed too large for intact removal may be broken down into a plurality of smaller pieces to facilitate removal from the internal body cavity. When severing tissue or otherwise treating the tissue, surgical smoke containing particulates is generated. The particulates may include hazardous chemicals and/or pathogens harmful to the clinician, staff, and/or the patient. The surgical smoke may also impair visualization and/or otherwise hinder a clinician.

### SUMMARY

As used herein, the term “distal” refers to the portion that is described which is further from a user, while the term “proximal” refers to the portion that is being described which is closer to a user. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent.

Further, any or all of the aspects described herein, to the extent consistent, may be used in conjunction with any or all of the other aspects described herein.

Provided in accordance with aspects of the present disclosure is a surgical smoke filtering device. The surgical smoke filtering device includes an annular collar defining a longitudinally-extending central passageway configured to be placed over an incision. A plurality of apertures configured to receive surgical smoke is disposed through an inner surface of the annular collar. The surgical smoke filtering device also includes a first electrode having a first electrical charge and a second electrode having a second electrical charge opposite the first electrical charge. The first electrode is disposed within a first annular channel defined by the annular collar and the second electrode is disposed within a second annular channel defined by the annular collar. The first and second electrodes are configured to generate an airflow to draw the surgical smoke through the annular collar for filtering particulates from the surgical smoke.

In an aspect of the present disclosure, the first and second electrodes are annular.

In another aspect of the present disclosure, the annular collar is configured to be coupled to an access device, which is configured to be at least partially inserted through the incision.

In still another aspect of the present disclosure, the first and second electrodes coaxially surround the longitudinally-extending central passageway.

In yet another aspect of the present disclosure, the first annular channel is in fluid communication with the plurality of apertures disposed through the inner surface of the annular collar.

In another aspect of the present disclosure, the first annular channel is in fluid communication with the second annular channel.

In still another aspect of the present disclosure, the first electrode is negatively charged, and the second electrode is positively charged.

In yet another aspect of the present disclosure, the first electrode is configured to apply a negative charge to the particulates contained within the surgical smoke, and the particulates negatively charged by the first electrode are configured to attach to the positively charged second electrode.

In another aspect of the present disclosure, the annular collar includes an outlet configured to operably couple to a source of suction.

In still another aspect of the present disclosure, the first and second annular channels coaxially surround the longitudinally-extending central passageway.

A surgical system provided in accordance with the present disclosure includes an access device configured to provide access to an internal surgical site through an incision and an annular collar defining a longitudinally-extending central passageway. The access device includes a proximal rim configured for positioning on an external side of the incision, a distal rim configured for positioning on an internal side of the incision, and a body extending between the proximal and distal rims. The body is configured to extend through the incision. The body also defines a passageway extending longitudinally therethrough to permit access to the internal surgical site through the incision. The annular collar is configured to be coupled to the proximal rim of the access device for filtering surgical smoke generated at the internal surgical site. The annular collar includes a first electrode having a first electrical charge and a second electrode having a second electrical charge opposite the first electrical charge. The first electrode is configured to apply the first electrical charge to particulates contained within the surgical smoke such that the particulates to which the first electrical charge is applied are configured to attach to a surface of the second electrode.

In an aspect of the present disclosure, the first and second electrodes are configured to generate an airflow to draw the surgical smoke through the annular collar.

In another aspect of the present disclosure, the annular collar includes a plurality of apertures disposed through an inner surface of the annular collar and configured to receive the surgical smoke therethrough.

In still another aspect of the present disclosure, the first electrode is negatively charged, and the second electrode is positively charged.

In still yet another aspect of the present disclosure, the first electrode is configured to apply a negative charge to particulates contained within the surgical smoke, and the particulates negatively charged by the first electrode are configured to attach to the positively charged second electrode.

In another aspect of the present disclosure, the first electrode is disposed within a first annular channel defined by the annular collar and the second electrode is disposed within a second annular channel defined by the annular collar.

In still another aspect of the present disclosure, the first and second annular channels coaxially surround the longitudinally-extending central passageway defined by the annular collar.

In still yet another aspect of the present disclosure, the first and second electrodes coaxially surround the longitudinally-extending central passageway defined by the annular collar.

In another aspect of the present disclosure, the longitudinally-extending central passageway defined by the annular collar is configured to longitudinally align with the passageway defined by the body of the access device upon coupling of the annular collar to the proximal rim of the access device.

Another surgical smoke filtering device provided in accordance with the present disclosure includes an annular collar defining a longitudinally-extending central passageway configured to be placed over an incision. A plurality of apertures configured to receive the surgical smoke therethrough is disposed through a surface of the annular collar. First and second electrodes are disposed within the annular collar and coaxially surround the longitudinally-extending central passageway. The first and second electrodes are configured to cause particulates contained within the surgical smoke to attach to one of the first or second electrodes to filter the particulates from the surgical smoke.

### BRIEF DESCRIPTION OF THE DRAWINGS

Various embodiments of the subject instrument are described herein with reference to the drawings wherein:

FIG. 1 is a perspective view illustrating a smoke filtering device provided in accordance with aspects of the disclosure;

FIG. 2 is a cross-sectional view, taken along cross-sectional line X-X shown in FIG. 1;

FIG. 3 is an exploded, top, perspective view of a system provided in accordance with the present disclosure including an access device and the smoke filtering device of FIG. 1;

FIG. 4 is a top, perspective view of the system of FIG. 3 in an assembled state; and

FIG. 5 is a top, perspective view of a system provided in accordance with the present disclosure including the access device of FIG. 3 and a smoke filtering device in accordance with another embodiment of the present disclosure.

### DETAILED DESCRIPTION

When dissecting a tissue specimen for extraction through an open incision, a significant amount of smoke plume may be generated within an internal surgical site and escape through the open incision and into the surrounding environment such as an operating room. The smoke plume may obstruct the surgical field, produce unpleasant odors, and/or release particulates into the operating room that are potentially harmful to the clinician, staff, and patient. For example, surgical smoke may contain hazardous chemicals such as mutagens and carcinogens, and/or pathogens such as HBV, HPV, HIV, etc. The present disclosure provides a smoke filtering device configured to be positioned external to a patient over an open incision for use during an open surgical procedure at an internal surgical site within a patient. The smoke filtering device houses a negatively charged electrode (e.g., an anode) and a positively charged electrode (e.g., a cathode). Utilizing the principles of ionization, the oppositely charged electrodes cooperate to generate an air flow or a so-called “ion flow” that serves to draw smoke through the smoke filtering device to filter potentially harmful particulates (e.g., chemicals, pathogens, etc.) from the smoke. As the smoke passes through the smoke filtering device, the anode negatively charges particulates carried by the smoke. Once negatively charged, the particulates are attracted to the surface of the positively charged cathode, which serves as a positively charged collector plate to which the negatively charged particulates attach prior to the smoke exiting the smoke filtering device. In this manner, harmful particulates (e.g., chemicals, pathogens, etc.) are trapped within the smoke filtering device such that smoke exiting the smoke filtering device is filtered and safe for releasing into the surrounding environment of the operating room.

The smoke filtering device may be adapted for use with an access device (e.g., wound protector, tissue guard, etc.) generally including a body extending between a proximal rim and a distal rim. The access device, an example of which is the SurgiSleeve™ Wound Protector sold by Medtronic, Inc., serves to maximize wound exposure while protecting the wound site from contamination and cutting during open surgery. During use of such an access device, the distal rim and body are inserted through an open incision in a patient such that the body and distal rim are positioned within the patient on an internal side of the open incision and the proximal rim is positioned outside of the patient on an external side of the open incision. The body defines a central passageway extending longitudinally therethrough to permit access to the surgical site through the open incision. As the longitudinally-extending passageway defined by the body is the only escape path for the smoke generated at the internal surgical site, positioning of the smoke filtering device over the open incision (e.g., via attachment to the access device) ensures that smoke escaping the surgical site through the body of the access device will be drawn into and through the smoke filtering device by the air flow cooperatively created by the oppositely charged electrodes within the smoke filtering device.

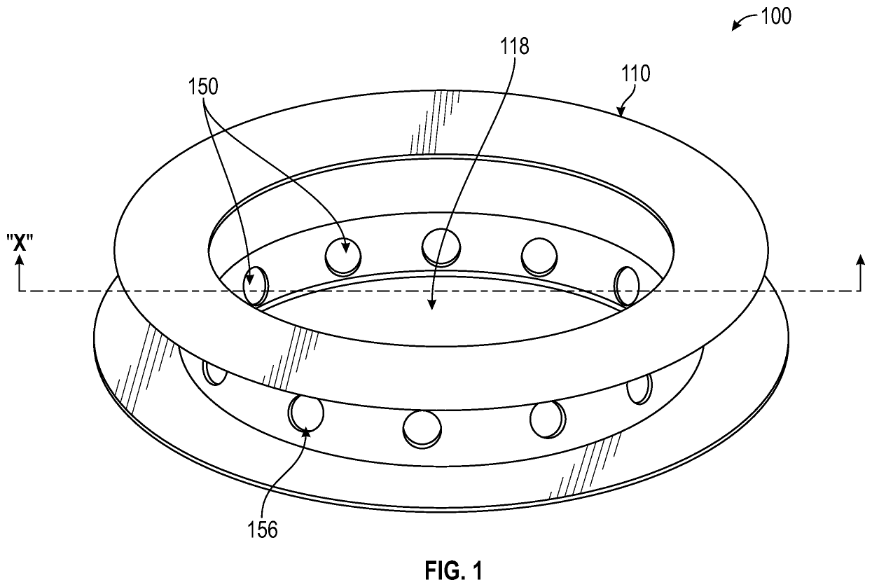

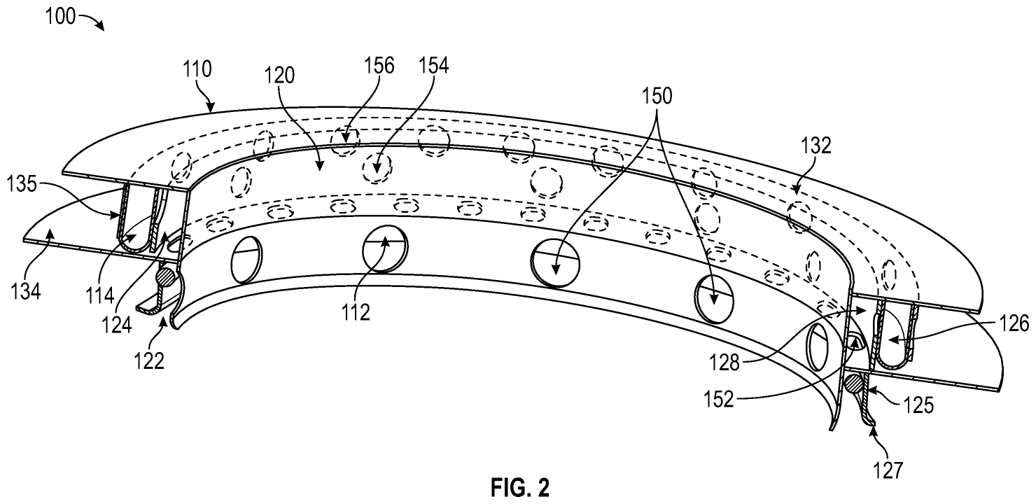

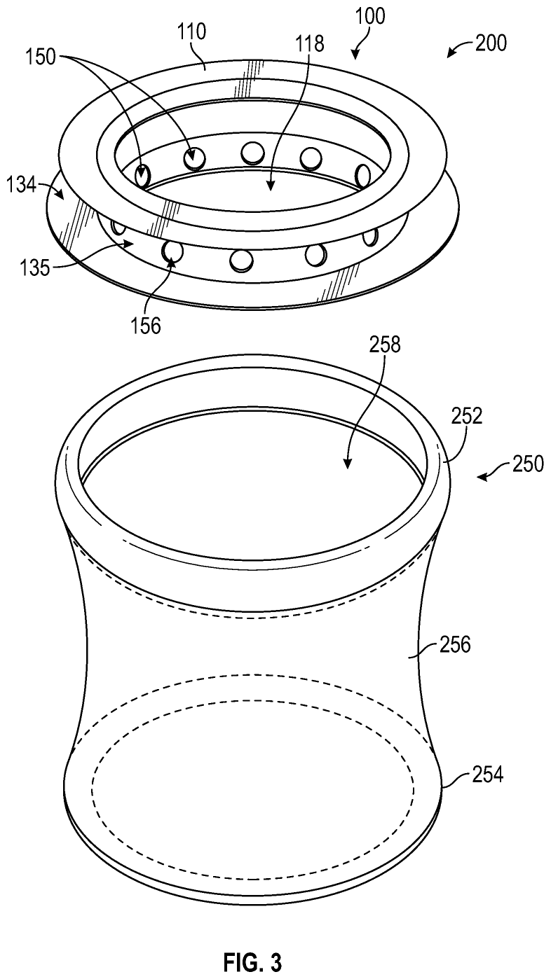

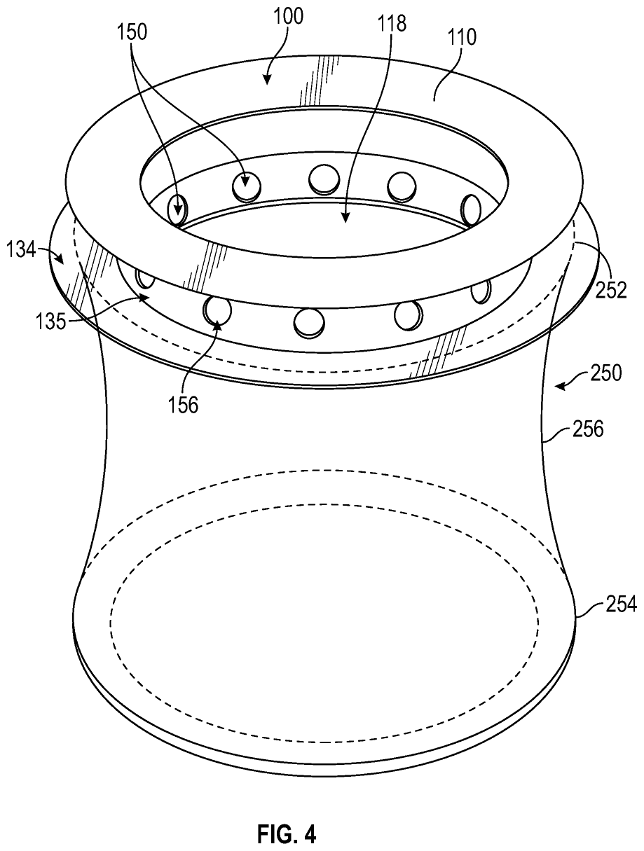

Referring to FIGS. 1 and 2, a smoke filtering device provided in accordance with the present disclosure is shown generally identified by reference numeral 100. Smoke filtering device 100 generally includes a collar 110 that houses a first annular electrode 112 and a second annular electrode 114 and defines an annular central passageway 118 configured to receive one or more surgical instruments (not shown) therethrough. An inner surface 120 of collar 110 extends circumferentially about central passageway 118, thereby defining an outer perimeter of central passageway 118. First annular electrode 112 is disposed within a distal annular channel 122 coaxially surrounding central passageway 118 and defined between inner surface 120 and an opposite distal outer surface 125. In embodiments, distal outer surface 125 defines a lip 127 that is configured to engage an underside of a proximal rim 252 of access device 250 to secure collar 110 to access device 250 (FIGS. 3 and 4).

Smoke filtering device 100 is formed from a suitable material, e.g., a biocompatible plastic such as, for example, polyethylene, polycarbonate, etc., from any suitable method, e.g., injection molding a single component or multiple components permanently secured or releasably engageable with one another. The material, thickness, and configuration of smoke filtering device 100 are selected such that smoke filtering device 100 is of sufficient stiffness to maintain its shape when coupled with an access device 250 (FIGS. 3 and 4). However, the material, thickness, and configuration of smoke filtering device 100 also provide sufficient resilient flexibility to permit manipulation of smoke filtering device 100 from an at-rest position for engagement with access device 250, with smoke filtering device 100 returning to or towards the at-rest position after engagement. Further, the material, thickness, and configuration of smoke filtering device 100 is selected such that smoke filtering device 100 is configured to withstand cutting and puncturing by surgical knives, scalpels, pencils, and the like, thereby protecting surrounding tissue and/or access device 250 from being cut or punctured. Smoke filtering device 100 may additionally or alternatively be configured to inhibit transfer of thermal and/or electrical energy therethrough to insulate surrounding tissue and/or access device 250 from thermal and/or electrical energy.

In embodiments of the present disclosure, first and second annular electrodes 112, 114 are oppositely charged relative to one another. For example, in one specific embodiment, first annular electrode 112 is negatively charged and second annular electrode 114 is positively charged. Utilizing the principles of ionization, the oppositely charged electrodes 112, 114 cooperate to generate an air flow that serves to draw smoke through smoke filtering device 100. As the smoke passes through smoke filtering device 100, first annular electrode 112 negatively charges particulates carried by the smoke. Once negatively charged, the particulates are attracted to the surface of the positively charged second annular electrode 114, which serves as a positively charged collector plate to which the negatively charged particulates attach. In this manner, potentially harmful particulates contained within the smoke are removed from the smoke and trapped within smoke filtering device 100 prior to the smoke exiting smoke filtering device 100.

Disposed proximal of distal annular channel 122 is an inner annular channel 124 coaxially surrounding central passageway 118 and an outer annular channel 126 coaxially surrounding inner annual channel 124 and central passageway 118. Inner annular channel 124 and outer annular channel 126 are defined between inner surface 120 and an opposite proximal outer surface 135 and are separated by a dividing surface 128 disposed coaxially between inner surface 120 and proximal outer surface 135. Inner annular channel 124 and outer annular channel 126 are disposed between a proximal lip 132 extending radially outward from inner surface 120 and a distal lip 134 extending radially outward from inner surface 120 and longitudinally spaced distal to proximal lip 132. Distal lip 134 separates distal annular channel 122 from proximal inner annular channel 124 and proximal outer annular channel 126, such that distal annular channel 122 is disposed distal to distal lip 134 and inner and outer annular channels 124, 126 are disposed proximal to distal lip 134. In embodiments, distal lip 134 engages proximal rim 252 of access device 250 upon coupling of smoke filtering device 100 to access device 250 (FIGS. 3 and 4).

With reference to FIG. 2, apertures defined through various surfaces of smoke filtering device 100 facilitate fluid communication between collar 110 and central passageway 118, between the various channels 122, 124, 126 of collar 110, and between collar 110 and the surrounding environment of the operating room. For example, a first plurality of apertures 150 is defined through a portion of inner surface 120 to place distal annular channel 122 in fluid communication with central passageway 118. A second plurality of apertures 152 is defined through a portion of distal lip 134 to place distal annular channel 122 in fluid communication with inner annular channel 124. Dividing surface 128 defines a third plurality of apertures 154 to place inner annular channel 124 in fluid communication with outer annular channel 126, and proximal outer surface 135 defines a fourth plurality of apertures 156 to place outer annular channel 126 in fluid communication with the surrounding environment of the operating room. During use of smoke filtering device 100, smoke from an internal surgical site escapes through an open incision around which collar 110 is placed and, as the smoke passes through central passageway 118, the smoke is drawn through one or more of apertures 150 into distal annular channel 122 where particulates contained within the smoke are electrically charged by first annular electrode 112. In embodiments of the present disclosure, first annular electrode 112 may operate as a negatively charged anode configured to apply a negative charge to particulates contained in smoke in proximity to first annular electrode 112 and second annular electrode 114 may operate as a positively charged cathode to which the negatively charged particulates are attracted. The attraction of the negatively charged particulates to the positively charged cathode induces an air flow or a so-called “ion flow” that serves to draw smoke through apertures 150 into distal annular channel 122 and subsequently into inner annular channel 124 toward the positively charged second annular electrode 114 disposed within outer annular channel 126. Particulates contained in the smoke attach to second annular electrode 114 prior to the smoke exiting outer annular channel 126 into the surrounding environment of the operating room.

With reference to FIGS. 3 and 4, a system 200 provided in accordance with the present disclosure includes smoke filtering device 100 and access device 250. Access device 250 may be configured as a tissue retractor, an access port, or other suitable access device configured for positioning within an opening in tissue, e.g., a surgical incision or a naturally-occurring orifice, to provide access therethrough into an internal surgical site. Access device 250 includes proximal rim 252 configured for positioning on an external side of the opening in tissue, a distal rim 254 configured for positioning on an internal side of the opening in tissue, and a body 256 extending between proximal and distal rims 252, 254, respectively. Body 256 is configured to extend through the opening in tissue and defines a passageway 258 extending longitudinally therethrough to permit access to an internal surgical site through the opening in tissue. At least a portion of body 256 of access device 250 may be flexible to facilitate insertion and positioning of access device 250 within the opening in tissue. In aspects, body 256 is formed from a flexible sleeve of material including one or more layers of material. Further, access device 250 may be selectively adjustable, e.g., by rolling proximal rim 254 distally about body 256, to retract tissue and/or secure access device 250 within the opening in tissue.

In use, access device 250 is positioned within an opening in tissue such that distal rim 254 is disposed on an internal surface of tissue on the internal side of the opening in tissue, body 256 extends through the opening in tissue, and proximal rim 252 is disposed on an exterior surface of tissue on the external side of the opening in tissue. Access device 250 may be adjusted to conform access device 250 to a patient's anatomy, retract tissue, and/or secure access device 250 within the opening in tissue.

With access device 250 disposed within the opening in tissue, smoke filtering device 100 is inserted into passageway 258 and lip 127 of distal outer surface 125 is flexed or otherwise manipulated to permit lip 127 to engage an underside of proximal rim 252, thereby securing smoke filtering device 100 to access device 250. With smoke filtering device 100 engaged within access device 250, surgical instrumentation may be inserted through central passageway 118 of smoke filtering device 100 into the internal surgical site to, for example, extract a tissue specimen therefrom. Smoke filtering device 100, as noted above, protects tissue as well as access device 250 during the insertion, manipulation, use and withdrawal of any such surgical instrumentation.

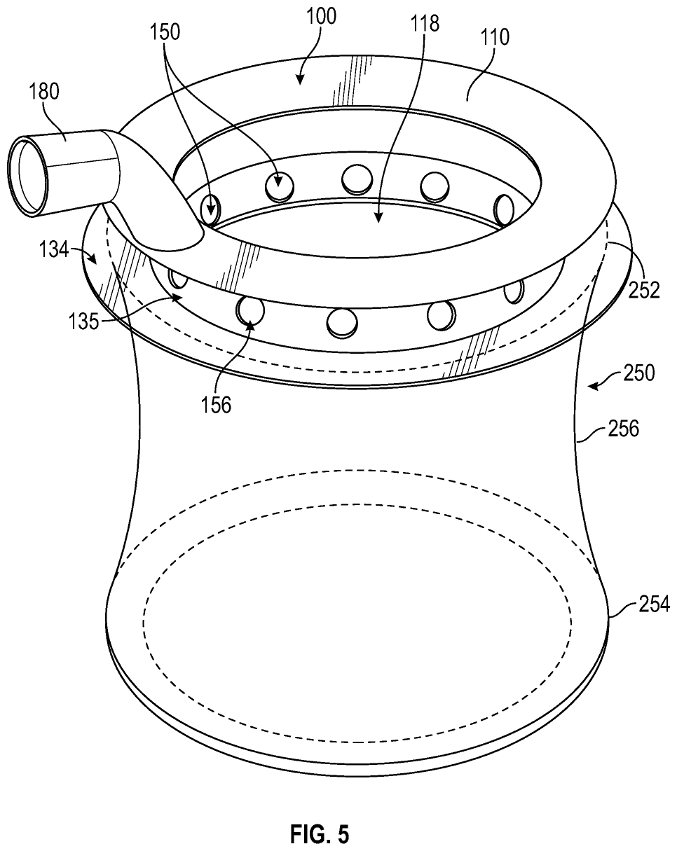

In some embodiments, it may be desirable to apply suction through smoke filtering device 100 to supplement the air flow created by the first and second annular electrodes 112, 114 for drawing smoke or other fluid from the internal surgical site into smoke filtering device 100. For example, FIG. 5 shows an embodiment of smoke filtering device 100 including an outlet 180 configured to be operably coupled to a source of suction (not shown) to establish suction through smoke filtering device 100. In some embodiments, apertures 156 defined through proximal outer surface 135 may be embodied as a single aperture (not shown) aligned with and in fluid communication with outlet 180 such that smoke is drawn from outer annular channel 126 directly into outlet 180 via a single aperture.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A surgical smoke filtering device, comprising:
an annular collar defining a longitudinally-extending central passageway configured to be placed over an incision;
a plurality of apertures disposed through an inner surface of the annular collar and configured to receive surgical smoke therethrough;
a first electrode disposed within a first annular channel defined by the annular collar, the first electrode having a first electrical charge; and
a second electrode disposed within a second annular channel defined by the annular collar and having a second electrical charge opposite the first electrical charge, the first and second electrodes configured to generate an airflow to draw the surgical smoke through the annular collar for filtering particulates from the surgical smoke.

2. The surgical smoke filtering device according to claim 1, wherein the first and second electrodes are annular.

3. The surgical smoke filtering device according to claim 1, wherein the annular collar is configured to be coupled to an access device, the access device configured to be at least partially inserted through the incision.

4. The surgical smoke filtering device according to claim 1, wherein the first and second electrodes coaxially surround the longitudinally-extending central passageway.

5. The surgical smoke filtering device according to claim 1, wherein the first annular channel is in fluid communication with the plurality of apertures disposed through the inner surface of the annular collar.

6. The surgical smoke filtering device according to claim 1, wherein the first annular channel is in fluid communication with the second annular channel.

7. The surgical smoke filtering device according to claim 1, wherein the first electrode is negatively charged, and the second electrode is positively charged.

8. The surgical smoke filtering device according to claim 7, wherein the first electrode is configured to apply a negative charge to the particulates contained within the surgical smoke, and the particulates negatively charged by the first electrode are configured to attach to the positively charged second electrode.

9. The surgical smoke filtering device according to claim 1, wherein the annular collar includes an outlet configured to operably couple to a source of suction.

10. The surgical smoke filtering device according to claim 1, wherein the first and second annular channels coaxially surround the longitudinally-extending central passageway.

11. A surgical system, comprising:
an access device configured to provide access to an internal surgical site through an incision, the access device including:
a proximal rim configured for positioning on an external side of the incision;
a distal rim configured for positioning on an internal side of the incision; and
a body extending between the proximal and distal rims and configured to extend through the incision, the body defining a passageway extending longitudinally therethrough to permit access to the internal surgical site through the incision; and
an annular collar defining a longitudinally-extending central passageway and configured to be coupled to the proximal rim of the access device for filtering surgical smoke generated at the internal surgical site, the annular collar including:
a first electrode having a first electrical charge; and
a second electrode having a second electrical charge opposite the first electrical charge, wherein the first electrode is configured to apply the first electrical charge to particulates contained within the surgical smoke such that the particulates to which the first electrical charge is applied are configured to attach to a surface of the second electrode.

12. The surgical system according to claim 11, wherein the first and second electrodes are configured to generate an airflow to draw the surgical smoke through the annular collar.

13. The surgical system according to claim 11, wherein the annular collar includes a plurality of apertures disposed through an inner surface of the annular collar and configured to receive the surgical smoke therethrough.

14. The surgical system according to claim 11, wherein the first electrode is negatively charged, and the second electrode is positively charged.

15. The surgical system according to claim 14, wherein the first electrode is configured to apply a negative charge to particulates contained within the surgical smoke, and the particulates negatively charged by the first electrode are configured to attach to the positively charged second electrode.

16. The surgical system according to claim 11, wherein the first electrode is disposed within a first annular channel defined by the annular collar and the second electrode is disposed within a second annular channel defined by the annular collar.

17. The surgical system according to claim 16, wherein the first and second annular channels coaxially surround the longitudinally-extending central passageway defined by the annular collar.

18. The surgical system according to claim 11, wherein the first and second electrodes coaxially surround the longitudinally-extending central passageway defined by the annular collar.

19. The surgical system according to claim 11, wherein the longitudinally-extending central passageway defined by the annular collar is configured to longitudinally align with the passageway defined by the body of the access device upon coupling of the annular collar to the proximal rim of the access device.

20. A surgical smoke filtering device, comprising:
an annular collar defining a longitudinally-extending central passageway configured to be placed over an incision;
a plurality of apertures disposed through a surface of the annular collar and configured to receive surgical smoke therethrough;
a first electrode disposed within the annular collar and coaxially surrounding the longitudinally-extending central passageway; and
a second electrode disposed within the annular collar and coaxially surrounding the longitudinally-extending central passageway, the first and second electrodes configured to cause particulates contained within the surgical smoke to attach to one of the first or second electrodes to filter the particulates from the surgical smoke.
